(19) United States
(12) Patent Application Publication
(10) Pub. No.: US 2021/0322632 A1
(43) Pub. Date: Oct. 21, 2021

(54) MODULAR SYNTHETIC TISSUE-GRAFT SCAFFOLD

(71) Applicant: OREGON HEALTH & SCIENCE UNIVERSITY, Portland, OR (US)

(72) Inventors: Luiz E. Bertassoni, Portland, OR (US); Avathamsa Athirasala, Portland, OR (US); Anthony Tahayeri, Sherwood, OR (US)

(21) Appl. No.: 17/263,873

(22) PCT Filed: Jul. 26, 2019
(86) PCT No.: PCT/US2019/043798

§ 371 Date: Jan. 27, 2021

Related U.S. Application Data
(60) Provisional application No. 62/711,422, filed on Jul. 27, 2018.

(51) Int. Cl. A61L 27/12; B33Y 80/00; B33Y 70/10; A61L 27/52; A61L 27/10; B29C 64/124; C12N 5/071

(52) U.S. Cl. CPC A61L 27/12; B33Y 80/00; B33Y 70/10; A61L 27/52; A61L 27/105; B29C 64/124; C12N 2501/155; A61L 2430/02; C12N 2513/00; C12N 2533/14; C12N 2533/18; C12N 2501/165; C12N 2501/135; C12N 5/0691

(57) ABSTRACT

A modular synthetic tissue-graft scaffold (10) includes one or more nominally identical scaffold cages (12) configured to facilitate regrowth of tissue of an organism in and around the scaffold cages. Each scaffold cage comprises a volumetric enclosure (18) bounded by a perforated wall structure (40). A recess (24) formed at one end of the volumetric enclosure defines an inner stepped coupling surface. An annular raised portion (26) positioned at the other end of the volumetric enclosure forms an outwardly projecting stepped seating surface sized to form a complementary matable surface to the inner stepped coupling surface for whenever an inner stepped coupling surface of another one of the cages is placed on the outer stepped seating surface of the scaffold cage. Corridors (46) extending through the perforated wall structure and communicating with passageways (54) within the volumetric enclosure enable migration of material within and out of the scaffold cage.

### RELATED APPLICATION

This application claims benefit of U.S. Patent Application No. 62/711,422, filed Jul. 27, 2018.

### FEDERALLY SPONSORED RESEARCH OR DEVELOPMENT

This invention was made with government support under Grant Number NIH R01 DE026170 awarded by the National Institutes of Health. The government has certain rights in the invention.

### COPYRIGHT NOTICE

© 2019 Oregon Health & Science University. A portion of the disclosure of this patent document contains material that is subject to copyright protection. The copyright owner has no objection to the facsimile reproduction by anyone of the patent document or the patent disclosure, as it appears in the Patent and Trademark Office patent file or records, but otherwise reserves all copyright rights whatsoever. 37 CFR § 1.71(d).

### TECHNICAL FIELD

Generally, the field involves methods for generating scaffold structures for tissue regeneration applications. More specifically, the field involves generation of modular scaffold cage that may be combined into larger scaffold structures. The modular scaffold cages are engineered to contain interconnected porous spaces into which a substrate such as a gel, including a hydrogel, may be introduced to produce a two-phase scaffold structure.

### BACKGROUND INFORMATION

Additive manufacturing (such as 3D-printing technology) has enabled significant progress in tissue-graft scaffold design and fabrication for regenerative medicine applications. This includes the capability to selectively fabricate patient-specific scaffolds of suitable shape, size, and three-dimensional complexity to support tissue regeneration of that patient's tissue defects. However, the integration of scaffold printing into the clinical workflow has been impeded by several technological challenges including the need for specialized equipment and personnel, poor scalability, lengthy printing time, as well as several post-printing steps necessary to make a scaffold compatible with patient implantation such as sterilization and post-polymerization. Accordingly, a patient-specific scaffold that is capable of selective, three-dimensional, on-site assembly that is scalable without the need of specialized equipment and personnel, yet ready for use in real-time, would allow patient-specific scaffolds to be integrated into the clinical workflow. Moreover, a preferable tissue-graft scaffold would be capable of being loaded with generative tissue-graft material such as cells, growth factors, hydrogels, microgels, or other therapeutics in a controllable and site-specific manner to enhance the spatial and temporal control of host tissue ingrowth within the grafted material.

Bone repair is one example of tissue regeneration that entails use of tissue-graft scaffolds. Approximately one in two adults is affected by some form of bone or musculoskeletal condition worldwide, which is twice the rate of heart and lung diseases. Craniotomies and other craniofacial procedures, such as vertical and horizontal bone augmentation, have an estimated cost of about $950 million each year, and it is estimated that more than 500,000 bone grafting procedures are conducted annually in the U.S. alone. Despite important limitations associated with autologous bone harvesting, such as the high hospitalization costs, and donor-site morbidity, bone autografts remain the gold-standard material to treat critical-sized bone defects. Therefore, it is frequently proposed that the ideal bone scaffold would match the key hallmarks of the native bone, while bypassing the challenges associated with its surgical extraction.

Although much progress has been made in the development of synthetic bone grafts, only 30% of treated patients regain function without the need for a secondary procedure, and graft failure rates can be as high as 50%. Autologous bone grafts are more successful compared to synthetic bone grafts due to their inherent vasculature, which is always present in autologous bone yet generally absent in synthetic scaffolds, and the failure of synthetic scaffolds to mimic the complexity of the cell-rich and nano-mineralized microenvironment that autologous bone provides. The native bone matrix consists of an osteocyte-laden, densely mineralized organic scaffold, where mineralization of ionic calcium and phosphorous is orchestrated on a nanometer scale, thereby resulting in a hierarchical architecture that is known to be key for bone's physical properties. Moreover, osteocytes embedded within this Calcium-and-Phosphorus-ion-rich (CaP-rich) milieu are known to control the process of bone remodeling from the “inside-out” and regulate its remodeling by secreting chemokines that attract host cells to the site of repair.

However, none of these key features are present in clinically available synthetic bone-graft scaffolds. Moreover, the reconstruction of large volume defects with autologous bone grafts remains a challenge; donor site morbidity limits the size of the harvested bone. Clinically available synthetic bone-graft scaffolds are typically composed of brittle pre-calcified ceramics or soft CaP-rich composites that rely on tissue ingrowth upon implantation for clinical success. Treatment of large volume defects with synthetic bone-graft scaffolds would ideally utilize a scaffold that is selectively scalable to the size and shape of the defect while having sufficient flexural strength to resist deformation after implantation.

### SUMMARY OF THE DISCLOSURE

The disclosed materials and methods relate to building models that are useful as scaffolds for tissue regeneration, such as for natural bone repair following trauma or surgery. Some of the disclosed embodiments use building blocks that are capable of forming a customized bone replacement scaffold for a portion of bone removed by surgery or trauma. A preferred modular synthetic tissue-graft scaffold includes a set of one or more nominally identical scaffold cages that are configured to facilitate regrowth of tissue of an organism in and around the scaffold cages. Each scaffold cage in the set comprises a volumetric enclosure that is bounded by a perforated wall structure and has interior and exterior surfaces and first and second opposite ends. The volumetric enclosure defines a central longitudinal axis that extends through the first and second opposite ends. The interior surface defines a boundary of an interior chamber of the volumetric enclosure, and the interior and exterior surfaces define between them a thickness of the perforated wall structure. A perforated platform set within the volumetric enclosure and in transverse relation to the central longitudinal axis forms a recess at the first end of the volumetric enclosure. The recess defines an inner stepped coupling surface that is bounded by the interior surface of the perforated wall structure. The perforated platform provides a passageway within the interior chamber of the volumetric enclosure between its first and second opposite ends. An annular raised portion is positioned at the second end of the volumetric enclosure and forms an outwardly projecting stepped seating surface. The stepped seating surface includes a first portion that is transverse to the central longitudinal axis and a second portion that is transverse to the first portion. The outwardly projecting stepped seating surface is sized to form a complementary matable surface to the inner stepped coupling surface whenever an inner stepped coupling surface of another one of the cages in the set is placed on the outer stepped seating surface of the scaffold cage. Corridors extending through the thickness of the perforated wall structure and communicating with the passageway within the interior chamber of the volumetric enclosure enable migration of material within and out of the scaffold cage.

A suitable synthetic bone-graft scaffold built with the disclosed scaffold cages may be made of a CaP-rich composite such as high-density β-tricalcium phosphate (β-TCP). Tissue-graft scaffolds fabricated with β-TCP have sufficient flexural strength to be printed as a permeable structure that nonetheless is resistant to deformation after implantation. Moreover, natural dissolution of the β-TCP post-implantation distributes osteoinductive, ionic calcium and phosphorous into the repair-site milieu while integrating the bone-graft scaffold into the surrounding tissue. A permeable β-TCP bone-graft scaffold is preferable for allowing the selective loading of tissue-graft material into the scaffold, and for allowing for movement of the tissue-graft material throughout the scaffold and host-insertion site, thus facilitating vascularization and tissue ingrowth within the bone-graft.

Additive manufacturing methods lend themselves well to fabricating micro-scale modular scaffolds. A modular scaffold enables significant scalability, allowing a user to employ as many scaffold modules as needed to fill the volume of a defect. A modular scaffold design also allows for a selective three-dimensional assembly of the scaffold to fit the three-dimensional shape of a defect. Moreover, a selective three-dimensional assembly utilizing micro-scale modules allows a user to employ a scaffold of heterogeneous tissue-graft material composition that is specific to the defect site and clinically meaningful.

Preferred embodiments of the disclosed tissue-graft scaffold fabricated by additive manufacturing methods, employing a modular and permeable structural design, loadable with microscale, site-specific, tissue-appropriate, tissue-graft materials are suitable for integrating patient-specific synthetic tissue-graft implants into the clinical workflow.

Additional aspects and advantages will be apparent from the following detailed description of preferred embodiments, which proceeds with reference to the accompanying drawings.

### BRIEF DESCRIPTION OF THE DRAWINGS

FIG. 1A is an exploded isometric view of four sets of scaffold cages arranged for assembly to construct a modular synthetic tissue-graft scaffold.

FIG. 1B is an isometric view of the four sets of scaffold cages of FIG. 1A after assembly that forms a three-dimensional synthetic tissue-graft scaffold.

FIG. 2A1 is an oblique isometric view showing a raised annular end, and FIG. 2A2 is an oblique isometric view showing an opposite, recessed end, of one embodiment of the disclosed scaffold cage.

FIG. 2B1 is a sectional view taken along lines 2B1-2B1 of FIG. 2A1, and FIG. 2B2 is a sectional view taken along lines 2B2-2B2 of FIGS. 2A2.

FIGS. 2C and 2D are plan views of, respectively, recessed and raised annular ends of the scaffold cage of FIGS. 2A1 and 2A2.

FIG. 3 shows a size and feature dimensions superimposed on a side elevation view of the scaffold cage of FIGS. 2A1 and 2A2.

FIG. 4 is a cross-sectional isometric view of a scaffold cage sheet formed by fusing in a 3×3 arrangement nine replicas of the scaffold cage of FIGS. 2A1 and 2A2.

FIG. 5A is a top plan view of six replicas of the scaffold cage of FIGS. 2A1 and 2A2, assembled in a 2×3 modular tissue-graft scaffold cage sheet.

FIG. 5B is a top plan view of six replicas of the scaffold cage of FIGS. 2A1 and 2A2, assembled in a 4×4 modular tissue-graft scaffold cage sheet.

FIG. 6 is a side elevation view of a three-dimensional synthetic tissue-graft scaffold assembled by coupling two 2×2 modular tissue-graft cage sheets formed from multiple replicas of the scaffold cage of FIGS. 2A1 and 2A2.

FIG. 7 is an isometric view of one embodiment of a 3×3 modular tissue-graft scaffold cage sheet configured to have scaffold cages oriented such that, respectively, not all of the annular raised portions and not all of the perforated platforms are positioned at first and second opposite ends of the volumetric enclosure.

FIGS. 8A, 8B, 8C, 8D, 8E, and 8F are pictorial views of three-dimensional synthetic tissue-graft scaffolds in six different three-dimensional shapes configured manually by a user.

FIGS. 9A, 9B, and 9C are diagrams showing side elevation views illustrating placement, in a diseased bone cavity, of a modular synthetic tissue-graft scaffold formed by assembly of sheets of different numbers of replicas of the scaffold cage of FIGS. 2A1 and 2A2 that are specially configured to facilitate bone regeneration.

FIG. 10 is a fragmentary oblique isometric view showing a raised annular end of a first alternative embodiment of the disclosed scaffold cage.

FIG. 11 is a fragmentary oblique isometric view showing a raised annular end of a second alternative embodiment of the disclosed scaffold cage.

### DETAILED DESCRIPTION OF PREFERRED EMBODIMENTS

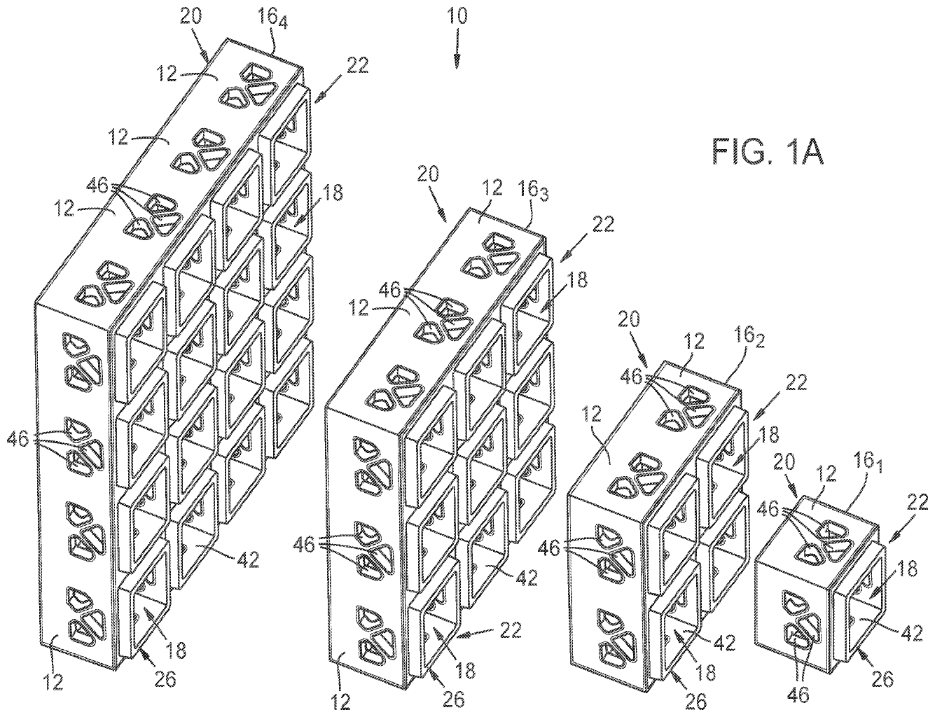

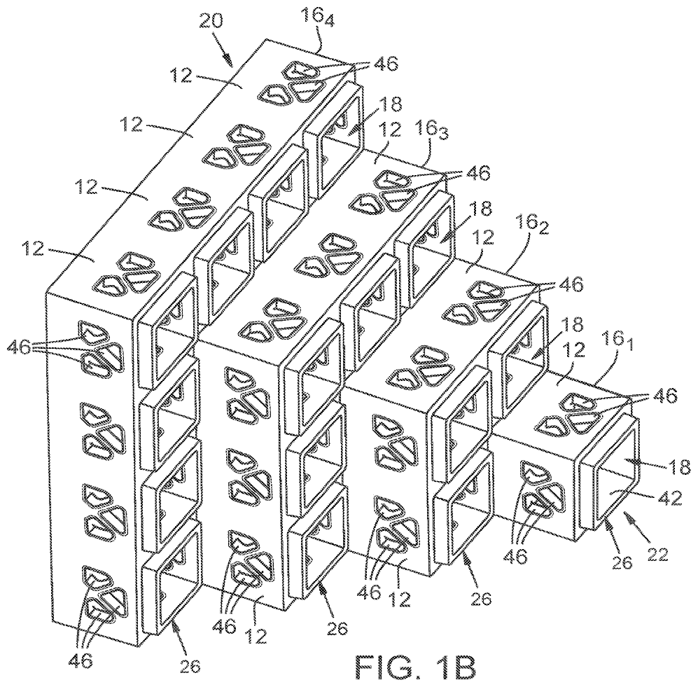

FIGS. 1A and 1B are respective exploded and isometric views of an example of a tissue-graft scaffold 10 that supports growth of tissue. Tissue-graft scaffold 10 is assembled with four sets of scaffold cages 12, each set in a different configuration from that of the other sets, including a single (1×1) scaffold cage 161, a 2×2 scaffold cage sheet 16₂, a 3×3 scaffold cage sheet 16₃, and a 4×4 scaffold cage sheet 16₄ (collectively, scaffold cage sheets 16). Each scaffold cage 12 in scaffold cage sheets 16 includes a volumetric enclosure 18 having a first opposite end 20 and a second opposite end 22. Volumetric enclosure 18 includes at its first end 20 a recess 24 and at its second end 22 an annular raised portion 26. Tissue-graft scaffold 10 is in the form of a three-dimensional shape that is implantable into a living site (not shown) by selectively inserting annular raised portions 26 into recesses 24 to couple sets of scaffold cages 12 together.

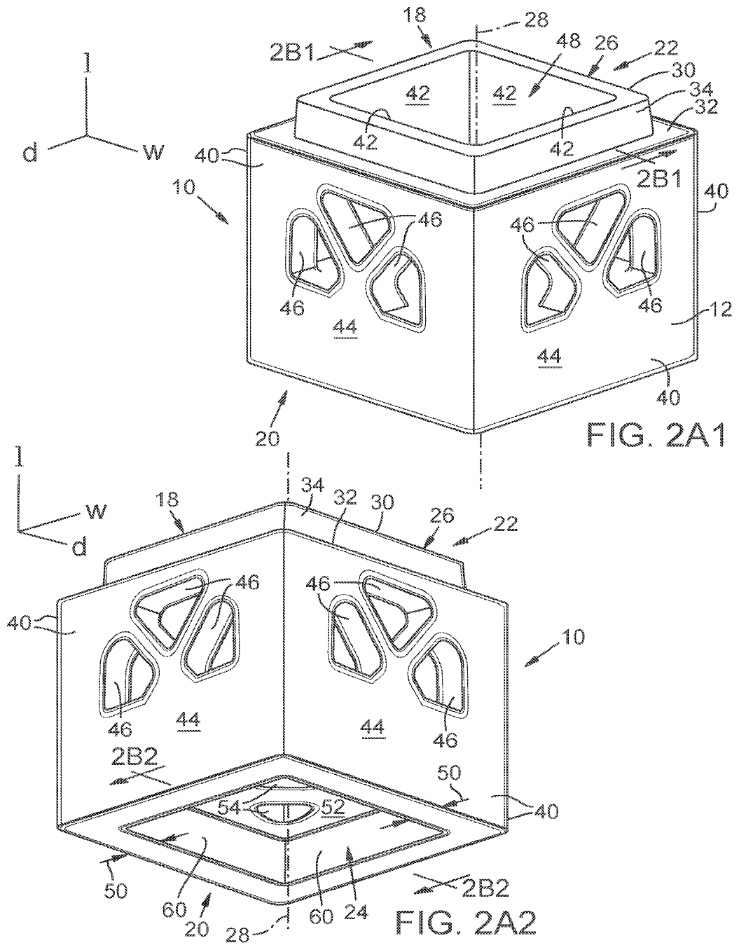

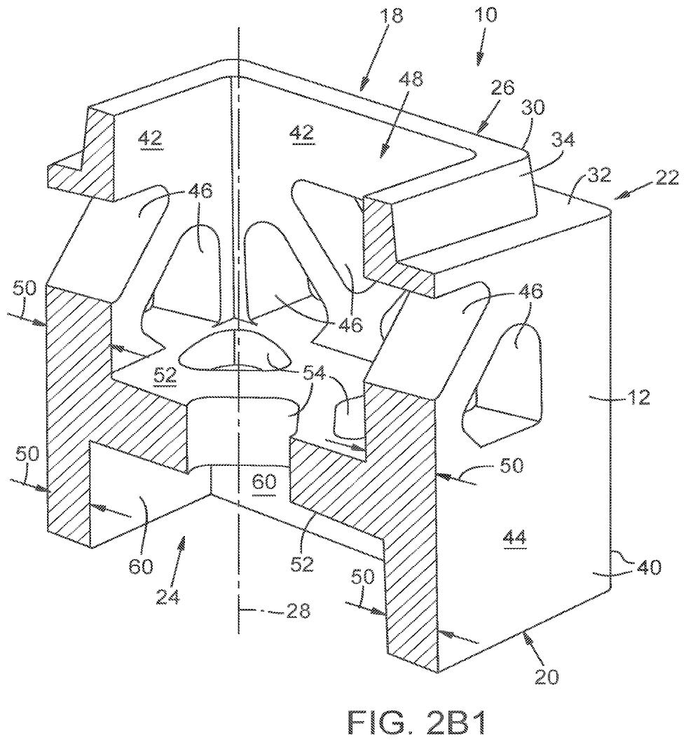

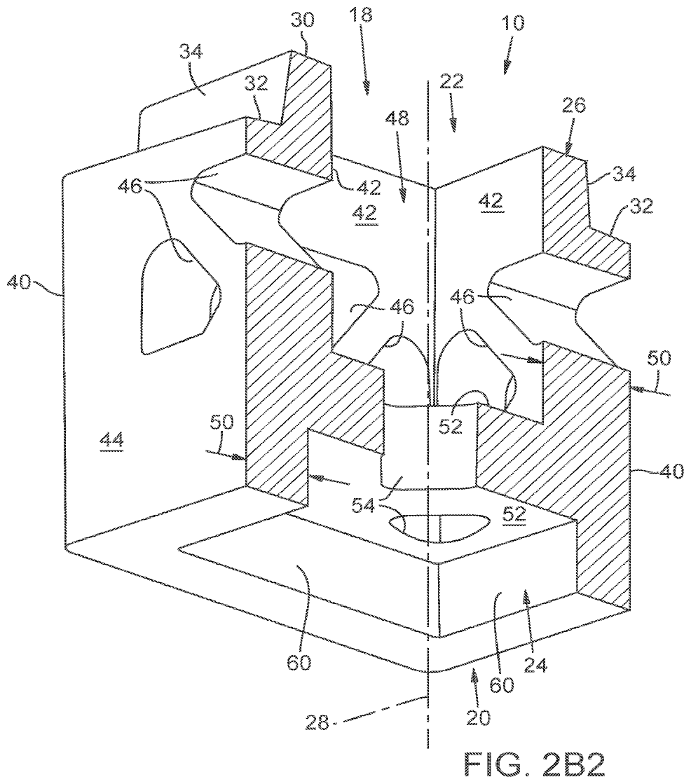

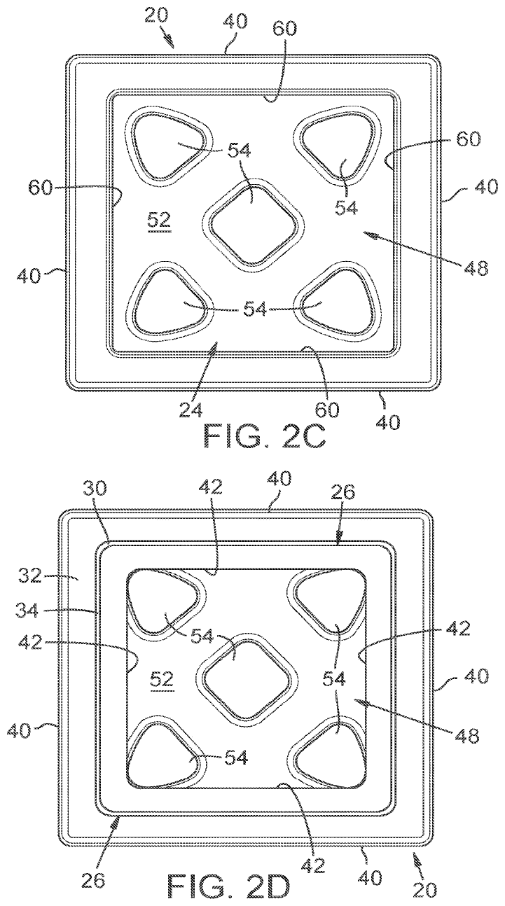

FIGS. 2A1 and 2A2, 2B1 and 2B2, 2C, and 2D are, respectively, isometric, sectional, bottom plan, and top plan views of a preferred embodiment of scaffold cage 12.

As shown in FIGS. 2A1 and 2A2, scaffold cage 12 includes volumetric enclosure 18 that defines a central longitudinal axis 28 extending through first end 20 and second end 22 and has recess 24 positioned at first end 20 and annular raised portion 26 positioned at second end 22. Annular raised portion 26 includes an outwardly stepped seating surface 30 that has a first portion 32 and a second portion 34, with the surface of first portion 32 set transverse to central longitudinal axis 28 and the surface of second portion 34 set transverse to the surface of first portion 32. Volumetric enclosure 18 is bounded by a perforated wall structure 40 to provide flexural strength to scaffold cage 12. Perforated wall structure 40 has an interior surface 42 and an exterior surface 44 that are connected by a set of corridors 46. Interior surface 42 defines an interior chamber 48 of sufficient volume to receive tissue-graft material, and interior surface 42 and exterior surface 44 define between them a thickness 50 of perforated wall structure 40. The measure of thickness 50 varies along the length of perforated wall structure 40. Corridors 46 extend through thickness 50 of perforated wall structure 40 to facilitate movement of tissue-graft material (not shown) throughout interior chamber 48, other scaffold cages, and host tissues.

In some embodiments, interior chamber 48 receives and contains tissue-graft material that supports growth of tissue. Examples of tissue-graft material include hydrogel, microgel, extracellular suspension, pharmaceutical compound, or autologous tissue. The tissue-graft material may be cell-laden or acellular. The tissue-graft material may contain cellular growth factors including Vascular Endothelial Growth Factor (VEGF), Platelet-Derived Growth Factor (PDGF), or Bone Morphogenic Protein 2 (BMP-2); be pre-vascularized; or be geometrically micropatterned.

FIGS. 2B1 and 2B2 show interior chamber 48, thickness 50 of perforated wall structure 40, corridors 46, and recess 24, the last of which has a length set by a perforated platform 52. Perforated platform 52 is set inwardly of second end 22 and in transverse relation to central longitudinal axis 28 to form recess 24 at first end 20 of volumetric enclosure 18. Perforated platform 52 provides support for tissue-graft material placed in interior chamber 48 and includes passageways 54 for the movement of tissue-graft material within interior chamber 48 between first end 20 and second end 22. Recess 24 defines an inner stepped coupling surface 60 bounded by interior surface 42. Inner stepped coupling surface 60 and outwardly stepped seating surface 30 are mutually sized to form complementary matable surfaces for coupling adjacent scaffold cages by inserting an outer stepped seating surface of an annular raised portion of one of the scaffold cages into a recess of the other scaffold cage.

In a preferred embodiment, the complementary matable surfaces form a friction-fit to secure the coupling of scaffold cages. “Friction-fit” is described herein as a fastening between two surfaces that is achieved by friction after the surfaces are joined together. In other embodiments, an adhesive may be used to fasten the complementary matable surfaces together.

FIG. 2C shows another view of recess 24, inner stepped coupling surface 60, perforated platform 52, passageways 54, and perforated wall structure 40. In the embodiment shown, recess 24 at first end 20 of volumetric enclosure 18 terminates at perforated platform 52. Perforated platform 52 provides passageways 54 for movement of tissue-graft material within interior chamber 48 between first end 20 and second end 22 of volumetric enclosure 18.

FIG. 2D shows another view of annular raised portion 26, outwardly stepped seating surface 30, interior chamber 48, perforated platform 52, passageways 54, and perforated wall structure 40. In the embodiment shown, annular raised portion 26 forming an outwardly projecting second end 22 of volumetric enclosure 18 is of rectangular (specifically, square) shape and forms an aperture that gives access to interior chamber 48 and perforated platform 52.

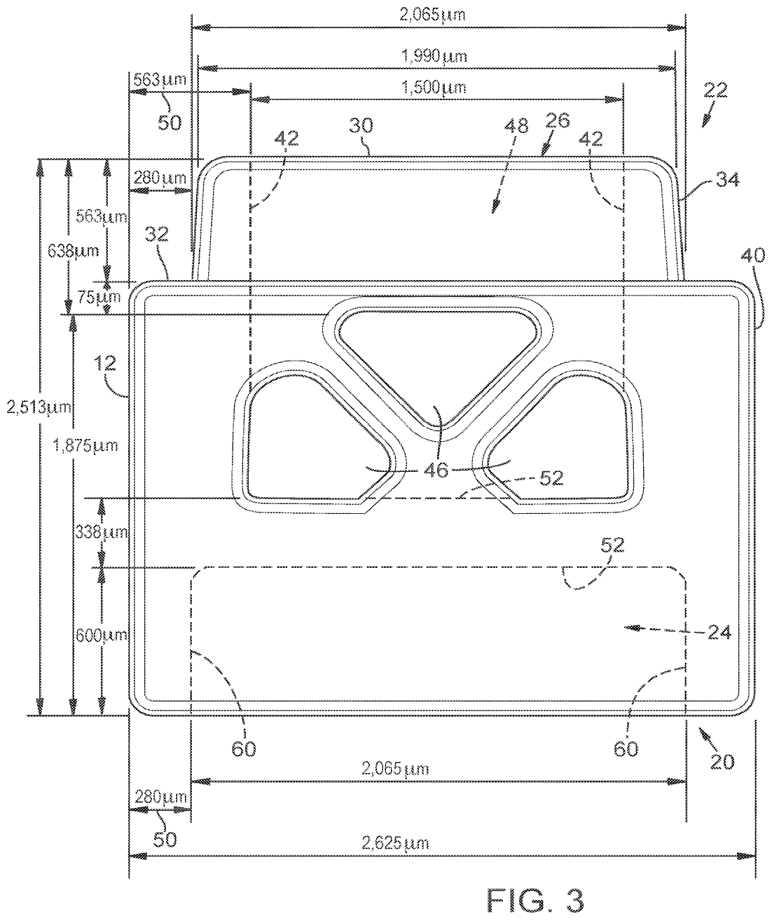

FIG. 3 is a schematic diagram showing size and feature dimensions superimposed on a side view of scaffold cage 12 of FIGS. 2A1 and 2A2. The length, width, and depth dimensions of scaffold cage 12 are defined by the coordinate system shown on FIGS. 2A1 and 2A2. In the embodiment shown, perforated wall structure 40 has a length of 1,950 μm, width of 2,625 μm, and depth of 2,625 μm, with thickness 50 of perforated wall structure 40 set to 563 μm and to 280 μm; perforated platform 52 has a thickness of 338 μm and is set to give recess 24 a length of 600 μm; annular raised portion 26 has a length of 563 μm, width of 2,625 μm, and depth of 2,625 μm, and has an aperture leading to interior chamber 48 of crosswise measure between about 1,500 μm-2,121 μm, with first portion 32 having a thickness of 225 μm; corridors 46 have apertures of crosswise measure between about 1 μm-800 μm and are set in perforated wall structure 40 at 75 μm from annular raised portion 26 and adjacent to thickness 50 and perforated platform 52.

In some embodiments, the dimensions of perforated wall structure 40 range between about (1,000 μm-3,000 μm)×(1,000 μm-3,000 μm)×(1,000 μm-3,000 μm), with thickness 50 ranging between about 100 μm-645 μm. In other embodiments, perforated platform 52 has a thickness ranging between about 125 μm-400 μm and is set to give recess 24 a length between about 225 μm-700 μm. In other embodiments, annular raised portion 26 has dimensions ranging between about (100 μm-645 μm)×(770 μm-3000 μm)× (770 μm-3000 μm) and an aperture leading to interior chamber 48 having a crosswise measure ranging between about 500 μm-2425 μm with first portion 32 having a thickness ranging between about 85 μm-320 μm. In further embodiments, corridors 46 have apertures having crosswise measures ranging between 190 μm-915 μm and are set in perforated wall structure 40 between 28 μm-86 μm from annular raised portion 26. These dimensional ranges are preferred to provide a therapeutically effective tissue-graft scaffold of sufficient flexural strength and permeability.

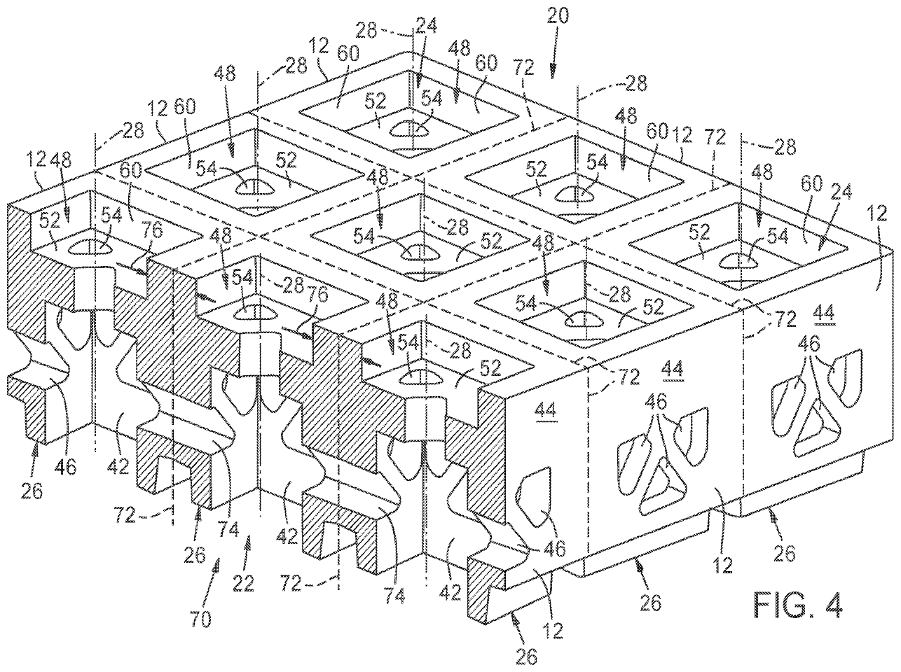

FIG. 4 is a cross-sectional isometric view of a scaffold cage sheet 70 formed by fusing in a 3×3 array nine nominally identical replicas of scaffold cage 12 shown in FIGS. 2A1 and 2A2. “Replicas” are described herein as nominally identical in that they exhibit the same features and dimensions within manufacturing tolerances. As shown in FIG. 4, each scaffold cage 12 in scaffold cage sheet 70 has a central longitudinal axis 28, an interior surface 42, an exterior surface 44, and an interior chamber 48. Scaffold cages 12 in scaffold cage sheet 70 are oriented such that their associated central longitudinal axes 28 are in generally parallel alignment and exterior surfaces 44 of mutually adjacent scaffold cages 12 are fused to each other to form a fused perforated wall structure 72 (shown in phantom lines) and a set of spatially aligned corridors 74. Interior surfaces 42 of mutually adjacent scaffold cages 12 define between them a thickness 76 of fused perforated wall structure 72. Spatially aligned corridors 74 extend through thickness 76 of fused perforated wall structure 72 to allow migration of tissue-graft material between interior chambers 48 of the mutually adjacent scaffold cages 12.

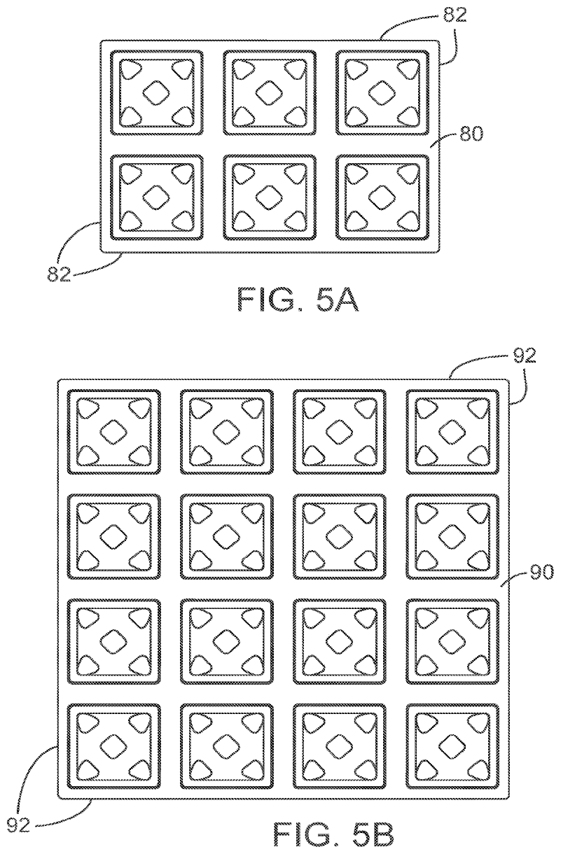

FIG. 5A is a top plan view of six replicas of the scaffold cage of FIGS. 2A1 and 2A2, assembled in a 2×3 modular tissue-graft scaffold cage sheet 80. In the embodiment shown, the six replicas of the scaffold cage of FIGS. 2A1 and 2A2 are fused to form a 2×3 fused perforated wall structure 82.

FIG. 5B is a top plan view of sixteen replicas of the scaffold cage of FIGS. 2A1 and 2A2, assembled in a 4×4 modular tissue-graft scaffold cage sheet 90. In the embodiment shown, the sixteen replicas of the scaffold cage of FIGS. 2A1 and 2A2 are fused to form a 4×4 fused perforated wall structure 92.

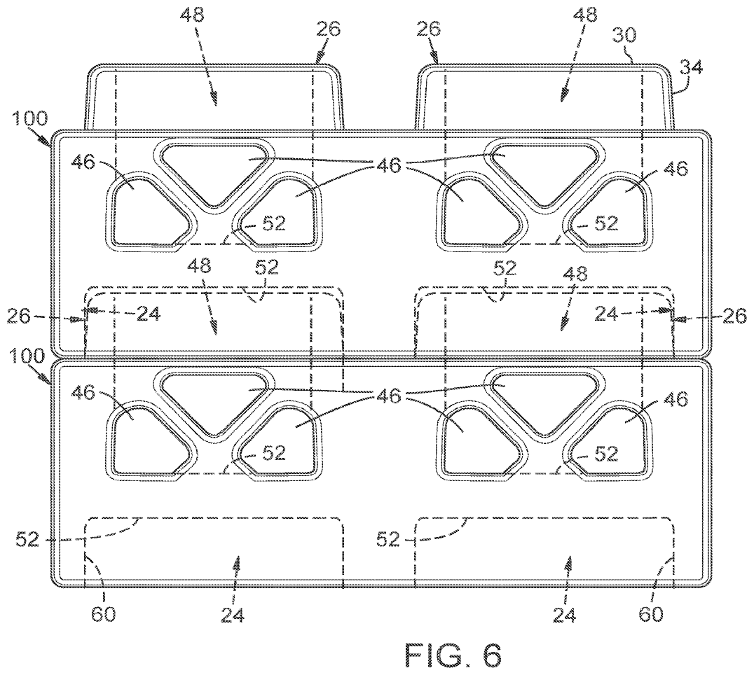

FIG. 6 is a side elevation view of a three-dimensional synthetic tissue-graft scaffold assembled by coupling two 2×2 modular tissue-graft cage sheets 100 formed from multiple replicas of the scaffold cage of FIGS. 2A1 and 2A2. FIG. 6 shows a set of annular raised portions 26 inserted into a set of recesses 24 to couple the two 2×2 modular tissue-graft cage sheets together. FIG. 6 shows, for each modular tissue-graft cage sheet 100, a set of annular raised portions 26, a set of corridors 50, and a fused perforated wall structure 94. Corridors 50 extend through a thickness of fused perforated wall structure 94 to a set of interior chambers 48 as shown in FIGS. 2A1 and 2A2 to facilitate movement of tissue-graft material (not shown) between interior chambers 48, other scaffold cages, and host tissues. In some embodiments, the complementary matable surfaces of annular raised portions 26 and recesses 24 may form a friction fit or be coupled by adhesive. FIG. 6 also shows, for each modular tissue-graft cage sheet 100, a set of interior chambers 48, a set of corridors 50, and a set of perforated platforms 52. Corridors 50 and perforated platforms 52 facilitate movement of tissue-graft material (not shown) between interior chambers 48 and host tissues as shown in FIGS. 2A1 and 2A2.

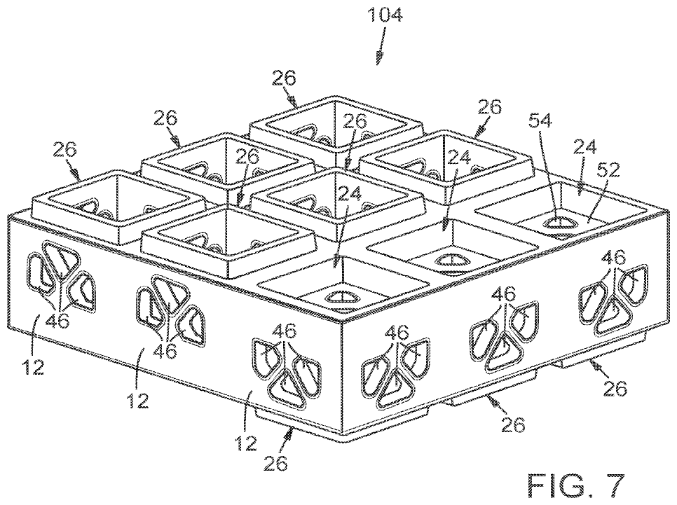

FIG. 7 is an isometric view of one embodiment of a 3×3 modular tissue-graft scaffold cage sheet 104 configured to have a set of scaffold cages 12 of FIGS. 2A1 and 2A2 including annular raised portions 26 positioned at first ends 20 and a set of perforated platforms 52 set to form a set of recesses 24 at second ends 22.

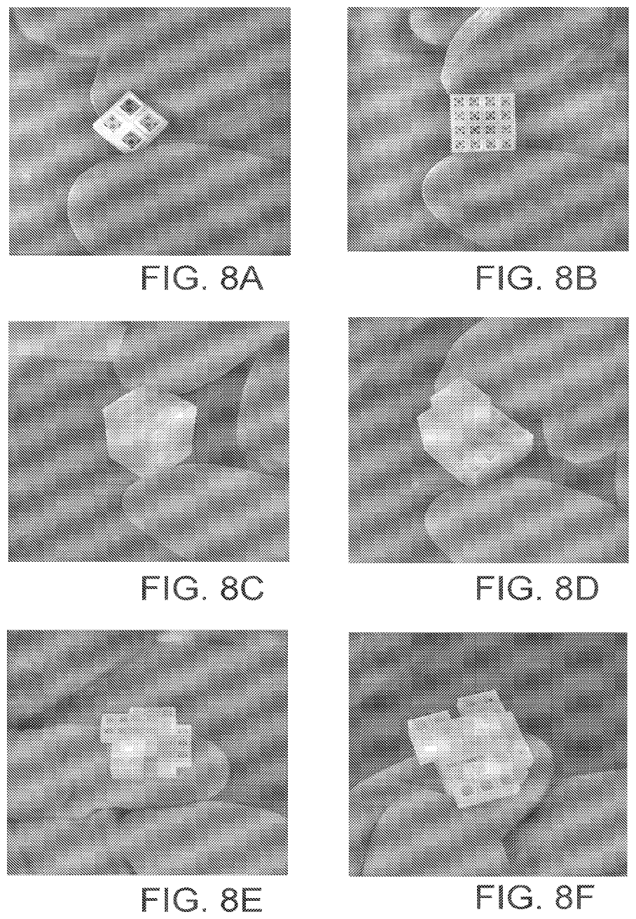

FIGS. 8A, 8B, 8C, 8D, 8E, and 8F are pictorial views of three-dimensional synthetic tissue-graft scaffolds in six different three-dimensional shapes configured manually by a user. Manual configuration of synthetic tissue-graft scaffolds by a user facilitates on-site, real-time construction of a tissue-graft scaffold of a three-dimensional shape suitable for insertion into the site of repair. On-site, real-time construction of tissue-graft scaffolds avoids the need to use x-ray, magnetic resonance, computed tomography, or ultrasound imaging methods to ascertain the three-dimensional shape of the site of repair.

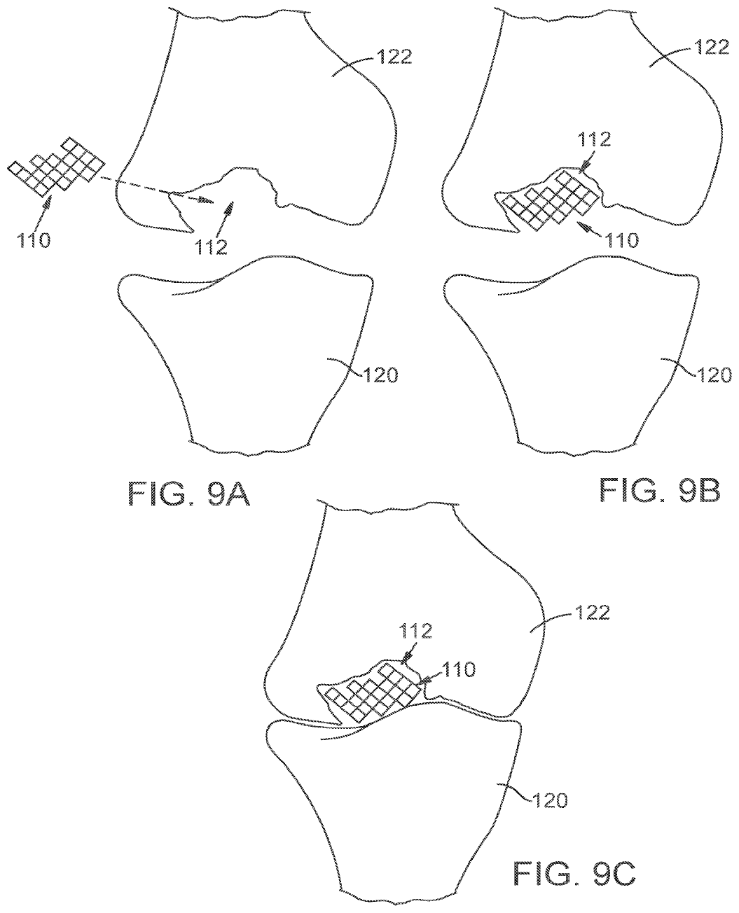

FIGS. 9A, 9B, and 9C are diagrams showing side elevation views illustrating placement of a modular synthetic tissue-graft scaffold 110 in a diseased bone cavity 112. Modular synthetic tissue-graft scaffold 110 is formed by assembly of multiple replicas of the scaffold cage of FIGS. 2A1 and 2A2 that are selectively configured to facilitate bone regeneration for placement in diseased bone cavity 112. FIG. 9A shows a lower, healthy bone 120 separated from an upper bone 122 having at its distal end diseased bone cavity 112. FIGS. 9B and 9C show placement of synthetic tissue-graft scaffold 110 in diseased bone cavity 112 with lower and upper bones 120 and 122, respectively, separated from and set adjacent to each other. (FIG. 9B showing, only for purposes of clarity, lower and upper bones 120 and 122 spaced part does not suggest that separation of them is required for placement of synthetic tissue-graft scaffold 110 into diseased bone cavity 112.) In the embodiment shown, synthetic tissue-graft scaffold 110 is configured to be of a three-dimensional shape that is suitable for insertion into diseased bone cavity 112.

In some embodiments, a cross-sectional surface area of the apertures of individual corridors 46 or spatially aligned corridors 74 ranges between about 10,000 μm²-810,000 μm² to allow vascularization to develop within the tissue-graft material. Corridors 46 or spatially aligned corridors 74 may be of any cross-sectional shape, including circular, elliptical, or polygonal; and the crosswise dimensions of the apertures of corridors 46 and spatially aligned corridors 74 may range between about 1 μm-1,000 μm.

In a preferred embodiment, the scaffold cages and scaffold cage sheets are made of β-tricalcium phosphates (β-TCP) for increasing the Ca²⁺/PO₄³⁻-dependent osteogenic signaling of human mesenchymal stem cells (hMSCs). LithaBone TCP 2000 (manufactured by Lithoz America LLC or “Lithoz”) is an example of a commercially prepared tri-calcium phosphate (Ca₃(PO₄)₂) product that is useful for bone replacement techniques. Moreover, tri-calcium phosphate materials generally are useful as bone replacement scaffolding because of their similarity to the mineral portion of human bone and have high biocompatibility, osteoconductivity, and resorbability. In some embodiments, the scaffold cages and scaffold cage sheets may be made from α-TCP, dicalcium phosphates, calcium carbonates, zirconium oxides or aluminum oxides. In other embodiments, they may be made of any material suitable for a specific function.

In a preferred embodiment, the scaffold cages and scaffold cage sheets are manufactured by lithography-based ceramic manufacturing (LCM) 3D printing technology. Examples of LCM 3D-printing instruments include the Lithoz Cera Fab 7500 and 8500 printers that have a printing resolution of about 40 μm. In one example of LCM 3D-printing, a ceramic powder (e.g., ASTM1088-04a certified β-TCP) is homogenously dispersed in a photocurable monomer and selectively polymerized via digital light projection (DLP) printing. The photolymerized slurry forms a composite of ceramic particles within a photopolymer matrix, and the organic matrix is removed via pyrolysis during sintering, which densifies the ceramic body to about 97% density. The resulting flexural strength of the printed material is about 35 MPa (similar to a trabecular bone), and its indentation modulus is generally equal to, or greater than, 100 GPa. In some embodiments, the scaffold cages and scaffold cage sheets may be manufactured using Osteoink™, which is a 3D-printable, osteoconductive calcium-phosphate material that sets in aqueous media without the need for sintering. In other embodiments, the scaffold cages and scaffold cage sheets may be manufactured by any other suitable three-dimensional printing technologies. In further embodiments, they may be made by any mold-based (such as reaction injection molding), sculpting-based, or subtractive manufacturing methods.

### Example

The following example further describes and demonstrates use of preferred embodiments of the disclosed tissue-graft scaffold 10. The example is given solely for the purpose of illustration and is not to be construed as limiting use of tissue-graft scaffold 10 because many variations thereof are possible without departing from the spirit and scope of uses of tissue-graft scaffold 10. This example demonstrates the benefits of the disclosed modular synthetic tissue-graft scaffold to repair a large-volume bone defect.

A patient is brought to surgery presenting with a large-volume open fracture. After an evaluation of the soft tissue and adequate debridement of the wound, the surgeon evaluates the open fracture of diseased bone cavity 112 of upper bone 122 as shown in FIG. 9 for the presence of vascularized and devascularized bone fragments. Any bone fragments that are completely devascularized are removed, leaving a dead space that must be closed.

After a study of the three-dimensional shape and size of the dead space remaining in diseased bone cavity 122, the surgeon selectively assembles multiple replicas of scaffold cage 12 as shown in FIGS. 2A1 and 2A2 to create synthetic tissue-graft scaffold 110 as shown in FIG. 9A. Synthetic tissue-graft scaffold 110 is fabricated with β-TCP as indicated for repair of a bone defect and then scaled to fill the volume of the dead space of diseased bone cavity 122 as shown in FIG. 9A. Interior chambers 48 of the multiple replicas of scaffold cage 12 constituting synthetic tissue-graft scaffold 110 contain cell-laden, geometrically micropatterned hydrogel constructs as bone-graft material. The hydrogel constructs are prevascularized and loaded with a mixture of VEGF, PDGF, and BMP-2 to facilitate and stimulate host cell migration and chemotaxis into interior chambers 48.

Prior to inserting synthetic tissue-graft scaffold 110 into the dead space of diseased bone cavity 122, the surgeon places a suture around synthetic tissue-graft scaffold 110 and ties a knot to ensure it does not come apart after implantation. A first attempt at insertion into diseased bone cavity 122, as shown in FIG. 9B, reveals that two replicas of scaffold cage 12 of a 2×3 scaffold cage sheet 80, as shown in FIG. 5A, used to assemble synthetic tissue-graft scaffold 110 are causing a poor fit. The surgeon removes synthetic tissue-graft scaffold 110 from bone cavity 122, shaves off the two poorly-fitting replica cages manually, and then reinserts synthetic tissue-graft scaffold 110 into the dead space of diseased bone cavity 122.

After determining that synthetic tissue-graft scaffold 110 suitably fits the dead space of diseased bone cavity 122, the surgeon secures synthetic tissue-graft scaffold 110 and the remaining vascularized host bone tissue together and closes the wound by suturing together the soft tissue surrounding diseased bone cavity 122 as represented by FIG. 9C.

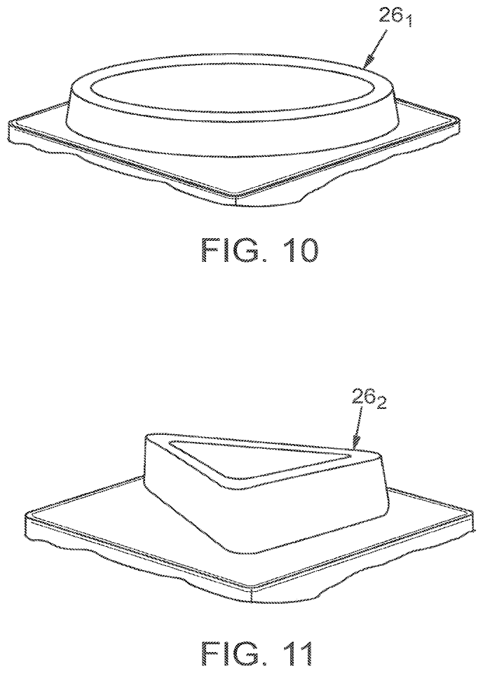

It will be obvious to those having skill in the art that many changes may be made to the details of the above-described embodiments without departing from the underlying principles of the invention. For example, annular raised portion 26 and recess 24 of a scaffold cage 12 can be of other than square shape. FIGS. 10 and 11 show alternative embodiments in which annular raised portions 26₁ and 26₂ are generally shaped as, respectively, an ellipse and a triangle. The associated recesses 24 of scaffold cages 12 of FIGS. 10 and 11 are of complementary shapes. Other possible shapes include other polygons or a circle. The scope of the present invention should, therefore, be determined only by the following claims.

1. In a modular, synthetic tissue-graft scaffold including a set of one or more nominally identical scaffold cages that are configured to facilitate regrowth of tissue of an organism in and around the scaffold cages, each scaffold cage in the set comprising:
a volumetric enclosure bounded by a perforated wall structure and having interior and exterior surfaces and first and second opposite ends, the volumetric enclosure defining a central longitudinal axis that extends through the first and second opposite ends, the interior surface defining a boundary of an interior chamber of the volumetric enclosure, and the interior and exterior surfaces defining between them a thickness of the perforated wall structure;
a perforated platform set within the volumetric enclosure and in transverse relation to the central longitudinal axis forms a recess at the first end of the volumetric enclosure, the recess defining an inner stepped coupling surface bounded by the interior surface of the perforated wall structure, the perforated platform providing a passageway within the interior chamber of the volumetric enclosure between its first and second opposite ends;
an annular raised portion positioned at the second end of the volumetric enclosure and forming an outwardly projecting stepped seating surface including a first portion that is transverse to the central longitudinal axis and a second portion that is transverse to the first portion, and the outwardly projecting stepped seating surface sized to form a complementary matable surface to the inner stepped coupling surface whenever an inner stepped coupling surface of another one of the cages in the set is placed on the outer stepped seating surface of the scaffold cage; and
corridors extending through the thickness of the perforated wall structure and communicating with the passageway within the interior chamber of the volumetric enclosure to enable migration of material within and out of the scaffold cage.

2. The modular scaffold of claim 1, in which the set includes an array of multiple nominally identical scaffold cages in the form of a scaffold cage sheet, the multiple scaffold cages oriented such that their associated central longitudinal axes are in generally parallel alignment and the exterior surfaces of mutually adjacent cages are fused to each other and thereby form a fused perforated wall structure, the fused perforated wall structure having a thickness through which spatially aligned corridors extend to allow migration of material between the interior chambers of the mutually adjacent scaffold cages.

3. The modular scaffold of claim 2, in which the scaffold cages forming the cage sheet include scaffold cages having annular raised portions positioned at first opposite ends and perforated platforms set to form recesses at second opposite ends.

4. The modular scaffold of claim 1, in which the modular scaffold is made of β-tricalcium phosphate.

5. The modular scaffold of claim 1, in which the modular scaffold is made of α-tricalcium phosphate, dicalcium phosphate, calcium carbonate, zirconium oxide or aluminum oxide.

6. The modular scaffold of claim 1, in which the modular scaffold is manufactured using a lithography-based three-dimensional printing technology.

7. The modular scaffold of claim 1, in which the modular scaffold is manufactured using a mold-based, a sculpting-based, or a subtractive manufacturing method.

8. The modular scaffold of claim 1, in which the inner stepped coupling surface is generally shaped as a circle, ellipse, or polygon.

9. The modular scaffold of claim 1, in which the annular raised portion is generally shaped as a circle, ellipse, or polygon.

10. The modular scaffold of claim 1, in which the perforated platform constitutes a first perforated platform, and further comprising a second perforated platform, the second perforated platform set transverse to the central longitudinal axis of the volumetric enclosure of the cage and proximal to the second end of the volumetric enclosure relative to the first perforated platform to define a platform pair, the platform pair providing a passageway within the interior chamber of the volumetric enclosure between the first and second ends.

11. The modular scaffold of claim 1, in which the exterior surface of the perforated wall structure constitutes one or more wall aspects, and the perforated wall structure includes no corridor extending through its thickness at one or more of the wall aspects.

12. The modular scaffold of claim 1, in which the passageway within the interior chamber terminates at and therefore does not extend through one of the first and second opposite ends of the volumetric enclosure.

13. The modular scaffold of claim 1, further comprising a tissue-graft material inserted into the interior chamber of the volumetric enclosure.
